(19) United States
(12) Patent Application Publication
(10) Pub. No.: US 2010/0286589 A1
(43) Pub. Date: Nov. 11, 2010

(54) Atherosclerotic Plaque Dissolution Composition

(76) Inventor: Kusai Saadeldin Aziz, Visalia, CA (US)

Correspondence Address: Ashok Tankha, 36 Greenleigh Drive, Sewell, NJ, 08080, US

(21) Appl. No.: 12/773,028

(22) Filed: May 4, 2010

Related U.S. Application Data
(60) Provisional application No. 61/175,458, filed on May 5, 2009.

(51) Int. Cl. A61K 31/015; A61M 37/00; A61K 31/047; A61K 31/20; A61K 31/01; A61P 9/10

(52) U.S. Cl. 604/20; 514/764; 514/738; 514/558; 514/762

(57) ABSTRACT

A method and chemical composition for modifying and dissolving atherosclerotic plaques formed in blood vessels of a patient is provided. The chemical composition comprising one or more of an organic substance, an inorganic substance, and a bioactive product is administered at sites of the plaques formed in the patient's blood vessels. The chemical composition comprises, for example, one or more of d-limonene, propylene glycol, octanic acid, 2-octane, and glycerine. The chemical composition enables modification of the plaques by altering composition of the plaques. The modification comprises partial dissolution, complete dissolution or elimination of the plaques. The modification makes the plaques amenable to different forms of plaque treatment, for example, balloon angioplasty, stenting, atherectomy, etc. The modified plaques are eliminated from the body of the patient. The modification or elimination of the plaques facilitates treatment of cardiovascular diseases caused due to plaques formed in the blood vessels of the patient.

### CROSS REFERENCE TO RELATED APPLICATIONS

This application claims the benefit of provisional patent application No. 61/175,458 titled “Atherosclerotic Plaque Dissolution Composition”, filed on May 5, 2009 in the United States Patent and Trademark Office.

The specification of the above referenced patent application is incorporated herein by reference in its entirety.

### BACKGROUND

The method and composition disclosed herein, in general, relates to plaque removal. More particularly, the method and composition disclosed herein relates to modifying, dissolving or eliminating plaques formed in blood vessels of a patient for treatment of cardiovascular diseases.

Atherosclerosis and plaque formation in blood vessels such as human arteries that cause cardiovascular diseases, for example coronary artery disease, myocardial infarction, carotid artery disease, strokes, and peripheral arterial disease are a common cause of human impairment and death.

Different treatment modalities, for example, balloon angioplasty, stents, atherectomy, and bypass surgery, have been developed to treat blockages caused by plaques. All these modalities are based on physical changes such as opening of a focal blockage or bypassing the blockages. However, these modalities do not address the pathological process of elimination of the plaques formed in the blood vessels.

Different physical methods typically used for treating plaques lead to the risk of adverse events such as death of the patient. For example, stents can lead to perforation of arteries and have certain incidences of restenosis of the arteries and thrombosis leading to myocardial infarctions and possibly death. Moreover, stents require use of blood thinners and increase the risk of bleeding.

Chemical dissolution methods have been used in dissolving gall bladder stones composed mainly of cholesterol but has not been implemented in atherosclerotic plaque dissolution.

Hence, there is long felt but unresolved need for a chemical composition and method that dissolves atherosclerotic plaques for treating cardiovascular diseases by eliminating plaques formed in the blood vessels of the patient suffering from cardiovascular diseases or by modifying the plaque characteristics to make the plaque more amenable to other physical methods of plaque treatment such as balloon angioplasty or stenting.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The chemical composition and method disclosed herein addresses the above stated need for treating cardiovascular diseases by modifying, dissolving, and eliminating plaques formed in the blood vessels of the patient suffering from cardiovascular diseases. A chemical composition comprising variable percentages of one or more of an organic substance, an inorganic substance, and a bioactive product is administered at sites of the plaques formed in the blood vessels of the patient. The chemical composition is administered at sites of plaque formation through, for example, a catheter. The chemical composition comprises, for example, one or more of about 20% to about 100% by weight of d-limonene, about 20% to about 100% by weight of propylene glycol, about 10% to about 98% by weight of octanic acid, about 10% to about 98% by weight of 2-octane, about 20% to about 100% by weight of glycerine, and a sufficient amount of a base composition to adjust the total weight percentage of the chemical composition to 100%. The base composition comprises, for example, water or a physiologic solution such as a saline solution, dextrose water, an organic solvent, etc.

In an embodiment, one or more additives may be added to the chemical composition disclosed herein. The additives are selected from a group comprising, for example, monoglyceride, monoctanoin, diethyl ether, halothane, ethanol, methanol, steroids, folic acid, heparin, octanediol, adenosine, high density lipoproteins (HDL), oils, etc. and any combination thereof. The chemical composition further comprises one or more optional medications, for example, direct thrombin inhibitors, thrombolytics, statins, antiplatelets, calcium channel blockers such as verapamil, etc. and any combination thereof. The chemical composition enables modification of the plaques by altering composition of the plaques in the blood vessels and thereby facilitating the removal of the modified plaques. The modification comprises partial dissolution, complete dissolution or elimination of the plaques. The modification makes the plaques amenable to different forms of plaque treatment, for example, balloon angioplasty, stenting, atherectomy, etc. The modified plaques are eliminated from the body of the patient. The modification or elimination of the plaques facilitates treatment of the cardiovascular diseases caused due to the plaques formed in the blood vessels of the patient.

The administered chemical composition is retained at the sites of plaque formation for a predetermined period of time to enable the modification of the plaques before removing the chemical composition by suctioning or flushing. The process of administering and removing the chemical composition may be repeated multiple times or may be continuous in nature, wherein the chemical composition is continuously delivered to the delivery site and at the same time suctioned or aspirated from such site. The chemical composition can be administered for plaque dissolution in different arterial blood vessels, for example, coronary arteries, peripheral arteries, arteries in lower extremities of the patient's body, renal arteries, carotid arteries, cerebral vessels, etc. The modification of the plaques by the administered chemical composition comprises partial dissolution, complete dissolution or modification of the plaques in the coronary arteries, the peripheral arteries, the arteries in the lower extremities of the patient's body, the renal arteries, the cerebral arteries, and cerebral vessels for enhancing luminal patency of these arterial blood vessels. The modification of the plaques by the administered chemical composition also comprises partial dissolution of the plaques for creating a channel for blood flow and for passing interventional equipment for different forms of plaque treatment.

In an embodiment, adjunctive methods are administered along with the chemical composition to obtain desired modification of the plaques. The adjunctive methods comprise utilizing low and high temperatures, pressure, radio magnetic waves, and/or physical agitation, etc.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein.

FIG. 1 illustrates a method for modifying and dissolving plaques formed in blood vessels of a patient.

FIG. 2 exemplarily illustrates a process flow for chemical dissolution of plaques formed in blood vessels of a patient.

### DETAILED DESCRIPTION OF THE INVENTION

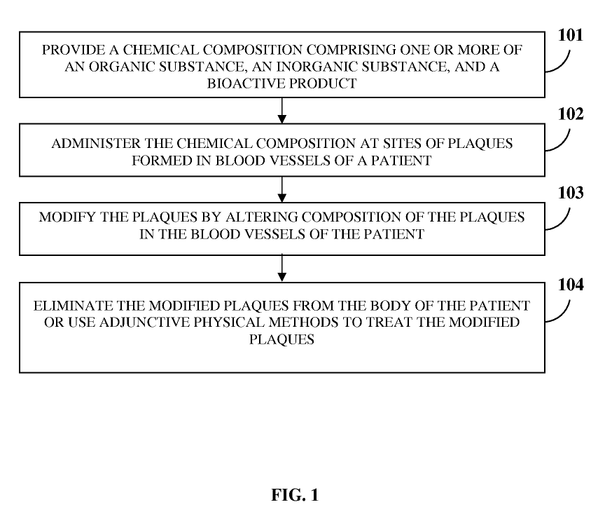

FIG. 1 illustrates a method for modifying and dissolving plaques formed in blood vessels of a patient. The method disclosed herein is used for treating cardiovascular diseases caused due to plaques formed in the blood vessels of the patient. Cardiovascular diseases are a class of diseases related to the heart or the blood vessels. The plaques are formed, for example, due to high levels of cholesterol in blood plasma. The plaques are also formed due to calcification. Calcification is the process of hardening of the tissues due to accumulation of mineral calcium in the tissues. Thrombosis is a pathological condition in which blood clots are formed within the blood vessels resulting in plaques. The plaques are, for example, atherosclerotic plaques. The atherosclerotic plaques are multiple plaques formed within the arteries.

A chemical composition, also referred to as a plaque dissolution composition, comprising one or more of an organic substance, an inorganic substance, and a bioactive product is provided 101. The bioactive product is, for example, a product that reacts with cells or tissues of a living body. The chemical composition is, for example, composed of a single chemical or multiple chemicals. The chemical composition comprises, for example, one or more of about 20% to about 100% by weight of d-limonene, about 20% to about 100% by weight of propylene glycol, about 10% to about 98% by weight of octanic acid, about 10% to about 98% by weight of 2-octane, about 20% to about 100% by weight of glycerine, and a sufficient amount of an optional base composition to adjust the total weight percentage of the chemical composition to 100%. The base composition comprises, for example, water or a physiologic solution such as a saline solution, dextrose water, an organic solvent, etc. or any combination thereof.

In an embodiment, the chemical composition comprises one or more additives for enhancing the function of the chemical composition. The additives are selected from a group comprising, for example, about 20% to about 100% by weight of d-limonene, about 10% to about 50% by weight of monoglyceride, about 1% to about 20% by weight of monoctanoin, about 1% to about 20% by weight of diethyl ether, about 1% to about 20% by weight of halothane, about 10% to about 50% by weight of ethanol, about 10% to about 50% by weight of methanol, about 5 milligrams (mg) to about 200 mg of steroids, about 1 mg to about 50 mg of folic acid, about 100 units to about 10000 units heparin, about 10% to about 50% by weight of octanediol, about 1 mg to about 50 mg of adenosine, about 5% to about 50% by weight of high density lipoproteins (HDL), about 5% to about 50% by weight of oils, etc. and any combination thereof. The chemical composition further comprises one or more optional medications, for example, direct thrombin inhibitors, thrombolytics, statins, antiplatelets, calcium channel blockers such as verapamil, etc. Different constituents of the chemical composition are directed towards different components of the plaques, for example, cholesterol, fibrous tissue, calcification, and thrombosis.

The physical nature of the chemical composition is generally liquid. However, the chemical composition may also contain certain gas components when administered into certain vascular beds such as the arteries in the lower extremities. The chemical composition disclosed herein is administered 102 at sites of the plaques formed in the blood vessels of the patient. The chemical composition is, for example, administered using delivery catheters to the sites of the plaques through the lumen of the artery to the inner surface or the luminal surface of the plaque. The catheters are tubes that can be inserted into a body cavity, for example, the blood vessels. The chemical composition is left in contact with the plaques for a certain period of time to exert chemical action on the plaques.

The chemical composition modifies 103 the plaques by altering composition of the plaques formed in the blood vessels of the patient. The chemical composition can be administered to selectively alter composition of the plaques formed in the blood vessels of the patient. The modification comprises partial dissolution, complete dissolution or elimination of the plaques. The modification makes the plaques amenable to different forms of plaque removal treatment, for example, balloon angioplasty, stenting, atherectomy, etc. Adjunctive methods, for example, heat, cold, low and high temperatures, pressure, radio magnetic waves, physical agitation, etc. are used any time before, during or after administration of the chemical composition to obtain desired modification of the plaques formed in the blood vessels of the patient. The chemical composition alters the plaque characteristic, that is, softens the plaque, when administered to the site of the plaques and hence is used as an adjunctive method for available interventional methods such as balloon angioplasty, stenting, and atherectomy. In an embodiment, the chemical composition disclosed herein is also used for treating chronic total occlusions of arteries in addition to variable degrees of atherosclerosis and luminal narrowing without chronic total occlusion. Chronic total occlusions are a severe form of atherosclerosis with total occlusion of the lumen of the artery. The chemical composition disclosed herein dissolves some of the atherosclerotic plaques and creates a channel for blood flow and for passing interventional equipment for different forms of plaque treatment, for example, balloon angioplasty, stenting, atherectomy, etc.

Furthermore, flushing or suction is used to remove pieces of the plaques. The process of administering and removing the chemical composition may be repeated multiple times or may be continuous in nature, wherein the chemical composition is continuously delivered to the delivery site and at the same time suctioned or aspirated from such site. Alternating flushing and suctioning may also be performed to maintain a constant exposure of the chemical composition to the luminal surface of the plaque by removing atherosclerotic materials that have already undergone alteration mediated by the chemical composition, and replacing the used chemical composition with fresh chemical composition in its original state.

The modified plaques are eliminated 104 from the body of the patient by a process of metabolism followed by excretion from the patient's body. The modified plaques are metabolized and excreted out of the body through different mechanisms and organs such as the liver and kidneys. The dissolution and purging of the plaque substance formed in the blood vessels widens the lumens of the treated arteries and therefore enables treatment of coronary artery disease, peripheral vascular disease, renal artery disease, etc. In an embodiment, adjunctive physical methods may be used 104 to treat the blood vessels having the modified plaques. The modification or elimination of the plaques formed in the blood vessels facilitates treatment of cardiovascular diseases caused due to the plaques formed in the blood vessels.

Balloon angioplasty involves mechanical widening of the blood vessels comprising plaques. Stenting is a procedure that involves use of a stent to open the arteries post angioplasty. Atherectomy is a process of reconstruction of the arteries after surgical removal of the plaques in the arteries. Filter wires are, for example, used in conjugation with the chemical composition to prevent distal embolization. Distal embolization is blockage of a blood vessel due to migration of a solid structure from one point of the blood vessel to another point in a direction of the blood stream.

The chemical composition facilitates partial dissolution, complete dissolution or modification of the plaques in the blood vessels of the heart muscle, that is, in the coronary arteries and enhances luminal patency of the coronary arteries, thereby decreasing incidence and severity of angina or myocardial infarctions also known as heart attacks.

Furthermore, the chemical composition facilitates partial dissolution, complete dissolution, or modification of the plaques in peripheral arteries, arteries in the lower extremities such as legs and feet, and enhances luminal patency of the arteries, thereby decreasing incidence and severity of peripheral vascular disease caused due to formation of plaques in the arteries of arms and legs, limbs ischemia, gangrene, etc. Limbs ischemia is the restricted flow of blood to limbs due to formation of plaques in the peripheral arteries. The chemical composition also facilitates partial dissolution, complete dissolution, or modification of the plaques in renal arteries and enhances luminal patency of the renal arteries, thereby decreasing incidence of different types of renal failure caused by reduced blood flow. Furthermore, the chemical composition facilitates partial dissolution, complete dissolution, or modification of the plaques in carotid arteries and other cerebral vessels and enhances luminal patency of the carotid arteries and other cerebral vessels, thereby decreasing incidence of certain types of strokes and certain neurological symptoms such as dizziness.

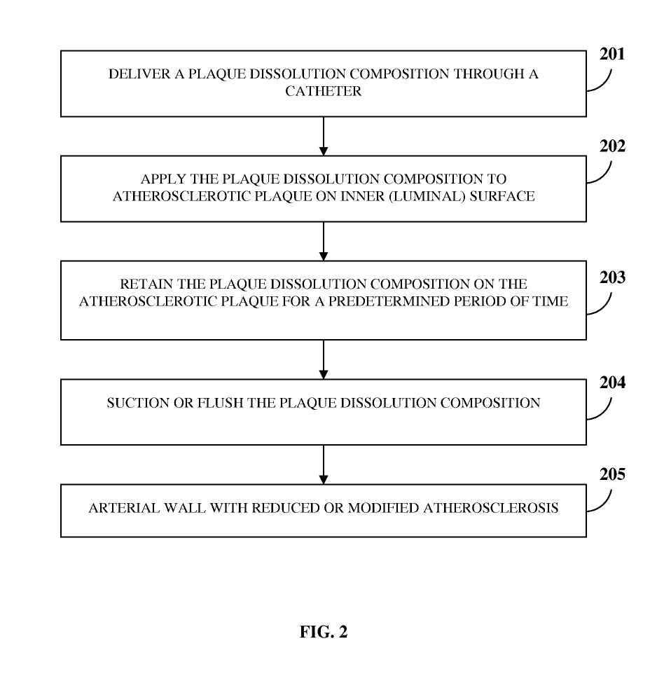

FIG. 2 exemplarily illustrates a process flow for chemical dissolution of plaques formed in blood vessels of a patient. The chemical composition herein referred to as a “plaque dissolution composition” is delivered 201 into the lumen of the affected blood vessels through a catheter. The plaque dissolution composition is applied 202 to the atherosclerotic plaques on the inner surface or the luminal surface within the blood vessels. The plaque dissolution composition is retained 203 on the atherosclerotic plaques for a predetermined period of time, for example, in a range of seconds or minutes to react with the atherosclerotic plaques. The plaque dissolution composition, including any dissolved plaques is removed from the affected sites of the arteries by suction or flushing 204 of the plaque dissolution composition. The plaque dissolution composition administered according to the method disclosed herein reduces or modifies 205 atherosclerosis of the arterial walls, and renders the atherosclerotic plaque amenable to other forms of plaque treatment.

The plaque dissolution composition and concentration is selected from biologically acceptable compounds or modified or controlled to be safe to biological tissues. Moreover, thorough testing is performed at different stages to obtain optimal compositions of the plaque dissolution composition. Research is performed by in-vitro testing of different concentrations of the above mentioned chemical compounds individually or in combination, followed by animal models prior to administering the plaque dissolution composition to humans. Furthermore, more than one set of the plaque dissolution composition is developed for different cases of cardiovascular diseases based on different factors, for example, vascular area, type of plaques, for example, according to the degree of calcification, and other characteristics of the patient. Several other chemical compositions can be used for the dissolution or elimination of the plaques according to the method disclosed herein, either alone or in combinations with the above mentioned chemical substances and products.

### Example 1

In an ex vivo experiment on atherosclerotic aorta tissue samples of a rabbit, significant dissolution of atherosclerotic plaque was noted after exposing the atherosclerotic plaque for about ten minutes to about thirty minutes to the following chemical composition: about 98% by weight of d-limonene and about 2% by weight of a base composition. A sufficient amount of an optional base composition, for example, another chemical compound, an organic solvent, water, saline solution, dextrose water, etc. or any combination thereof was added to the chemical composition to adjust the total weight percentage of the chemical composition to 100%.

### Example 2

In another ex vivo experiment on atherosclerotic aorta tissue samples of a rabbit, significant dissolution of atherosclerotic plaque was noted after exposing the atherosclerotic plaque for about ten minutes to about thirty minutes to the following chemical composition: about 99.9% by weight of propylene glycol and about 0.1% by weight of a base composition. A sufficient amount of an optional base composition, for example, another chemical compound, an organic solvent, water, saline solution, dextrose water, etc. or any combination thereof was added to the chemical composition to adjust the total weight percentage of the chemical composition to 100%.

### Example 3

In another ex vivo experiment on atherosclerotic aorta tissue samples of a rabbit, significant dissolution of atherosclerotic plaque was noted after exposing the atherosclerotic plaque for about ten minutes to about thirty minutes to the following chemical composition: about 98% by weight of octanic acid and about 2% by weight of a base composition. A sufficient amount of an optional base composition, for example, another chemical compound, an organic solvent, water, saline solution, dextrose water, etc. or any combination thereof was added to the chemical composition to adjust the total weight percentage of the chemical composition to 100%.

### Example 4

In another ex vivo experiment on atherosclerotic aorta tissue samples of a rabbit, significant dissolution of atherosclerotic plaque was noted after exposing the atherosclerotic plaque for about ten minutes to about thirty minutes to the following chemical composition: about 98% by weight of 2-octane and about 2% by weight of a base composition. A sufficient amount of an optional base composition, for example, another chemical compound, an organic solvent, water, saline solution, dextrose water, etc. or any combination thereof was added to the chemical composition to adjust the total weight percentage of the chemical composition to 100%.

### Example 5

In another ex vivo experiment on atherosclerotic aorta tissue samples of a rabbit, significant dissolution of atherosclerotic plaque was noted after exposing the atherosclerotic plaque for about ten minutes to about thirty minutes to the following chemical composition: about 100% by weight of glycerine. A sufficient amount of an optional base composition, for example, another chemical compound, an organic solvent, water, saline solution, dextrose water, etc. or any combination thereof was added to the chemical composition to adjust the total weight percentage of the chemical composition to 100%.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

I claim:

1. A method for modifying and dissolving plaques formed in blood vessels of a patient, comprising:
providing a chemical composition comprising one or more of an organic substance, an inorganic substance, and a bioactive product;
administering said chemical composition at sites of said plaques formed in said blood vessels of said patient; and
modifying said plaques by altering composition of said plaques in said blood vessels by said chemical composition, wherein said modification comprises one of partial dissolution, complete dissolution and elimination of said plaques, wherein said modification makes said plaques amenable to different forms of plaque treatment.

2. The method of claim 1, wherein said chemical composition comprises one or more of:
about 20% to about 100% by weight of d-limonene;
about 20% to about 100% by weight of propylene glycol;
about 10% to about 98% by weight of octanic acid;
about 10% to about 98% by weight of 2-octane;
about 20% to about 100% by weight of glycerine; and
a sufficient amount of a base composition to adjust total weight percentage of said chemical composition to 100%.

3. The method of claim 1, wherein said chemical composition comprises one or more additives selected from a group comprising about 20% to about 100% by weight of said d-limonene, about 10% to about 50% by weight of monoglyceride, about 1% to about 20% by weight of monoctanoin, about 1% to about 20% by weight of diethyl ether, about 1% to about 20% by weight of halothane, about 10% to about 50% by weight of ethanol, about 10% to about 50% by weight of methanol, about 5 mg to about 200 mg of steroids, about 1 mg to about 50 mg of folic acid, about 100 units to about 10000 units of heparin, about 10% to about 50% by weight of octanediol, about 1 mg to about 50 mg of adenosine, about 5% to about 50% by weight of high density lipoproteins, about 5% to about 50% by weight of oils, and any combination thereof.

4. The method of claim 1, wherein said chemical composition comprises medications comprising one or more of direct thrombin inhibitors, thrombolytics, statins, antiplatelets, calcium channel blockers, and any combination thereof.

5. The method of claim 4, wherein said calcium channel blockers comprise verapamil.

6. The method of claim 1, wherein said chemical composition is administered at sites of formation of said plaques in said blood vessels of said patient through a catheter.

7. The method of claim 1, wherein said administered chemical composition is retained at said sites of formation of said plaques for a predetermined period of time to enable said modification of said plaques before removing said chemical composition by one of suctioning and flushing.

8. The method of claim 1, wherein said modification of said plaques by said administered chemical composition comprises one of partial dissolution, complete dissolution and modification of said plaques in coronary arteries for enhancing luminal patency of said coronary arteries.

9. The method of claim 1, wherein said modification of said plaques by said administered chemical composition comprises one of partial dissolution, complete dissolution and modification of said plaques in peripheral arteries and arteries in lower extremities of a body of said patient for enhancing luminal patency of said peripheral arteries and said arteries of said lower extremities.

10. The method of claim 1, wherein said modification of said plaques by said administered chemical composition comprises one of partial dissolution, complete dissolution and modification of said plaques in renal arteries for enhancing luminal patency of said renal arteries.

11. The method of claim 1, wherein said modification of said plaques by said administered chemical composition comprises one of partial dissolution, complete dissolution and modification of said plaques in carotid arteries and cerebral vessels for enhancing luminal patency of said carotid arteries and said cerebral vessels.

12. The method of claim 1, wherein said modification of said plaques by said administered chemical composition comprises partial dissolution of said plaques for creating a channel for blood flow and for passing interventional equipment for said different forms of said plaque treatment.

13. The method of claim 1, further comprising administering adjunctive methods comprising utilizing one or more of low and high temperatures, pressure, radio magnetic waves, and physical agitation to obtain desired modification of said plaques.

14. A chemical composition for modifying and dissolving atherosclerotic plaque in blood vessels of a patient, comprising one or more of:
about 20% to about 100% by weight of d-limonene;
about 20% to about 100% by weight of propylene glycol;
about 10% to about 98% by weight of octanic acid;
about 10% to about 98% by weight of 2-octane;
about 20% to about 100% by weight of glycerine; and
a sufficient amount of a base composition to adjust total weight percentage of said chemical composition to 100%;
whereby administering said chemical composition at sites of said atherosclerotic plaque modifies said atherosclerotic plaque by altering composition of said atherosclerotic plaque formed in said blood vessels.

15. The chemical composition of claim 14, further comprising one or more of about 10% to about 50% by weight of monoglyceride, about 1% to about 20% by weight of monoctanoin, about 1% to about 20% by weight of diethyl ether, about 1% to about 20% by weight of halothane, about 10% to about 50% by weight of ethanol, about 10% to about 50% by weight of methanol, about 5 mg to about 200 mg of steroids, about 1 mg to about 50 mg of folic acid, about 100 units to about 10000 units of heparin, about 10% to about 50% by weight of octanediol, about 1 mg to about 50 mg of adenosine, about 5% to about 50% by weight of high density lipoproteins, about 5% to about 50% by weight of oils, and any combination thereof.

16. The chemical composition of claim 14, wherein said base composition comprises one of water, dextrose water, a saline solution, and an organic solvent.

17. A chemical composition for modifying and dissolving plaques in blood vessels of a patient, comprising one or more of:
about 10% to about 100% by weight of one or more of an organic substance, an inorganic substance, and a bioactive product, wherein said organic substance, said inorganic substance, and said bioactive product are selected from a group comprising d-limonene, propylene glycol, octanic acid, 2-octane, and glycerine; and
about 0% to about 90% by weight of one or more additives selected from a group comprising d-limonene, monoglyceride, monoctanoin, diethyl ether, halothane, ethanol, methanol, steroids, folic acid, heparin, octanediol, adenosine, high density lipoproteins, oils, and any combination thereof;
whereby administering said chemical composition at sites of said plaques formed in said blood vessels of said patient modifies said plaques by altering composition of said plaques in said blood vessels.

18. The chemical composition of claim 17, wherein said d-limonene is about 20% to about 100% by weight of said chemical composition.

19. The chemical composition of claim 17, wherein said propylene glycol is about 20% to about 99.9% by weight of said chemical composition.

20. The chemical composition of claim 17, wherein said octanic acid is about 10% to about 98% by weight of said chemical composition.

21. The chemical composition of claim 17, wherein said 2-octane is about 10% to about 98% by weight of said chemical composition.

22. The chemical composition of claim 17, wherein said glycerine is about 20% to about 100% by weight of said chemical composition.

23. The chemical composition of claim 17, wherein said one or more additives are selected from a group comprising about 20% to about 100% by weight of said d-limonene, about 10% to about 50% by weight of said monoglyceride, about 1% to about 20% by weight of said monoctanoin, about 1% to about 20% by weight of said diethyl ether, about 1% to about 20% by weight of said halothane, about 10% to about 50% by weight of said ethanol, about 10% to about 50% by weight of said methanol, about 5 mg to about 200 mg of said steroids, about 1 mg to about 50 mg of said folic acid, about 100 units to about 10000 units of said heparin, about 10% to about 50% by weight of said octanediol, about 1 mg to about 50 mg of said adenosine, about 5% to about 50% by weight of said high density lipoproteins, about 5% to about 50% by weight of said oils, and any combination thereof.

24. The chemical composition of claim 17, further comprising optional medications comprising one or more of direct thrombin inhibitors, thrombolytics, statins, antiplatelets, calcium channel blockers, and any combination thereof.

25. The chemical composition of claim 17, further comprising a sufficient amount of a base composition to adjust total weight percentage of said chemical composition to 100%, wherein said base composition comprises one of water, dextrose water, a saline solution, an organic solvent, and any combination thereof.
